(19) United States
(12) Patent Application Publication
(10) Pub. No.: US 2015/0209265 A1
(43) Pub. Date: Jul. 30, 2015

(54) SPHERICAL FORMS OF CROSS-LINKED HYALURONIC ACID AND METHODS OF USE THEREOF

(71) Applicant: Allergan Holdings France S.A.S., Courbevoie (FR)

(72) Inventor: Kenneth N. Horne, San Francisco, CA (US)

(21) Appl. No.: 14/604,017

(22) Filed: Jan. 23, 2015

Related U.S. Application Data
(60) Provisional application No. 61/932,164, filed on Jan. 27, 2014.

(51) Int. Cl. A61K 8/73; A61Q 19/08

(52) U.S. Cl. CPC A61K 8/735; A61Q 19/08; A61K 2800/41; A61K 2800/91

(57) ABSTRACT

This disclosure relates generally to spherical forms comprising cross-linked hyaluronic acid, methods of making such spherical forms and using thereof, for example, in aesthetic applications (e.g., facial contouring, soft tissue augmentation products) and the like.

### RELATED APPLICATION

This application claims priority to U.S. Provisional Patent Application No. 61/932,164, filed Jan. 27, 2014, the entire disclosures of which is incorporated herein by this specific reference.

### FIELD

This disclosure relates generally to spherical forms of hyaluronic acid, methods of making such spherical forms and uses thereof, for example, in aesthetic applications (e.g., facial contouring, dermal filling), and the like.

### BACKGROUND

Hyaluronic acid (HA) is a linear polysaccharide (i.e., non-sulfated glycosaminoglycan) consisting of a repeated disaccharide unit of alternately bonded β-D-N-acetylglucosamine and β-D-glucuronic acid which can be depicted by the formula:

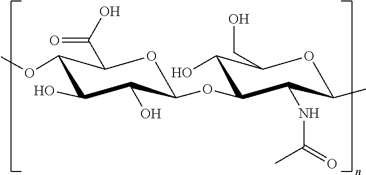

where n is the number of repeating units. Hyaluronic acid is sometimes referred to by the nomenclature (-4GlcUAβ1-3GlcNAcβ1-)ₙ) and is a chief component of the extracellular matrix found, for example, in connective, epithelial and neural tissue. Natural hyaluronic acid is highly biocompatible because of its lack of species and organ specificity and is often used as a biomaterial in tissue engineering and as a common ingredient in soft tissue augmentation products.

Natural hyaluronic acid has poor in vivo stability due to rapid enzymatic degradation and hydrolysis and, accordingly, various chemically modified forms of hyaluronic acid (e.g., cross-linked forms, ionically modified forms, esterified forms, etc.) have been synthesized to address this problem. Currently, hyaluronic acid or cross-linked versions thereof are used in various gel forms, for example as soft tissue augmentation products, adhesion barriers, and the like.

However, issues exist with the use of gels of hyaluronic acid or its cross-linked versions as soft tissue augmentation products. First, the force required to dispense gels of hyaluronic acid or its cross-linked versions is non-linear which can cause an initial ejection of a “glob” of gel that many physicians report when using injectable hyaluronic acid gels. Second, precisely dispensing hyaluronic gels to specific locations can be difficult because such gels have little mechanical strength. Further, the gel will occupy the space of least resistance which makes its use in many applications (e.g., treatment of fine wrinkles) problematic as the gel will often migrate into unintended spatial areas rendering the cosmetic procedure difficult and possibly even dangerous. Third, many common soft tissue augmentation products which are injected into the treatment site as a liquid or a gel, such as Restylane® (hyaluronic acid), Juvederm® (hyaluronic acid) Radiesse® (calcium hydroxyl apatite), Sculptra® (poly-L-lactic acid) and Perlane® (hyaluronic acid), are capable of migration and/or causing unsightly “lumps” which are painful to treat. Fourth, these soft tissue augmentation products are not recommended for use around the eyes as migration from the injection site can cause blindness, tissue necrosis, and in rare cases even stroke. Clinicians also find performing lip augmentations using these fillers time consuming and patients find treatments in this area so painful that nerve blocks are routinely performed.

Accordingly, spherical forms of hyaluronic acid and its cross-linked versions have been developed to address the issues associated with gels of hyaluronic acid.

### SUMMARY

It is contemplated that the new spherical forms offer improvements in the delivery, in both method and time, as well as placement of substantially cross-linked hyaluronic acid compositions. Spherical forms allow clinicians to more precisely implant cross-linked hyaluronic acid compositions in areas that may be difficult to implant of other types of hyaluronic acid forms.

In one embodiment, for example, a spherical form comprises substantially cross-linked hyaluronic acid. The spherical form, for example, may contain hyaluronic acid that is substantially cross-linked with at least about 15 mole % of a butanediol diglycidyl ether (BDDE) derivative relative to the repeating disaccharide unit of the hyaluronic acid, and at least about 5% noncross-linked hyaluronic acid relative to the weight of total hyaluronic acid solids.

In one embodiment, herein provides for a method of treating a wrinkle in a subject in need thereof. In such an aspect, the plurality of spherical forms are inserted into the skin of a patient adjacent to or under the wrinkle. The plurality of spherical forms are then applied under the wrinkle, thereby treating the wrinkle. In one embodiment, upon exposure to body fluids or by manually hydrating, the plurality of spherical forms expand upon hydration and such expansion is typically sufficient to fill-in the wrinkle. It is advantageous to have a spherical form expand upon hydration because the invasiveness of the insertion profile is minimized, however, spherical forms designed to not expand can also be used to treat the wrinkle.

In another embodiment, there is provided a method of providing facial contouring in a subject in need thereof. In this embodiment, the plurality of spherical forms are inserted into the skin at or adjacent to the desired treatment location, e.g., the lips, the nasolabial fold, the tear trough, etc. The plurality of spherical forms are then applied thereby providing facial contouring. In one embodiment, the spherical forms may be applied to various planes of the dermal tissue.

Also encompassed is a kit of parts comprising a plurality of spherical forms. In some embodiments, the kit further comprises a means for delivering the plurality of spherical forms. The means for delivery may include a syringe or needle.

In still other aspects, methods of using a plurality of spherical forms of hyaluronic acid as soft tissue augmentation products, facial contouring, and the like is provided. These embodiments, as well as others, are discussed in more detail below.

### BRIEF DESCRIPTION OF THE DRAWINGS

Certain aspects are best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 shows a schematic of hyaluronic acid cross-linked with butanediol diglycidyl ether (BDDE).

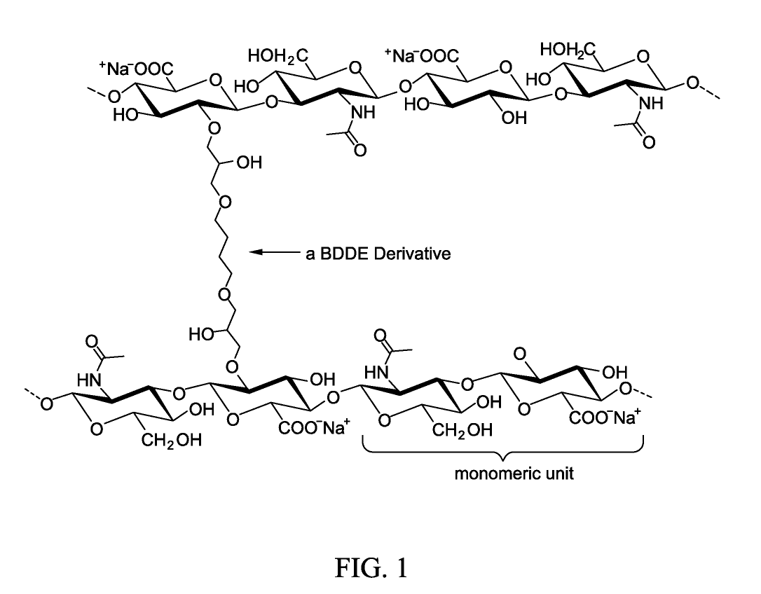

FIGS. 2A-2C show treatment of a wrinkle. FIG. 2A illustrates a cross-sectional view of a fold or a wrinkle; FIG. 2B illustrates a spherical form implanted beneath a wrinkle that is not yet hydrated; and FIG. 2C illustrates a spherical form implanted beneath a wrinkle that is fully hydrated and has flattened the surface appearance of the wrinkle.

### DETAILED DESCRIPTION

Described herein are spherical forms of substantially cross-linked hyaluronic acid, methods for their preparation and uses thereof and to specific shapes formed there from. However, the following terms will first be defined.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “a spherical forms” includes a plurality of spherical forms.

### 1. Definitions

Unless defined otherwise, all technical and scientific terms used herein have the same meaning as commonly understood by one of ordinary skill in the art to which this disclosure belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) claimed. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

The term “hyaluronic acid” or “HA” refers to the polymer having the formula:

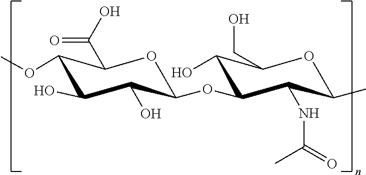

where n is the number of repeating units. All sources of hyaluronic acid are useful, including bacterial and avian sources. Hyaluronic acids useful have a molecular weight of from about 0.5 MDa (mega Dalton) to about 3.0 MDa. In some embodiments, the molecular weight is from about 0.6 MDa to about 2.6 MDa and in yet another embodiment, the molecular weight is from about 1.4 MDa to about 1.7 MDa. In some embodiments, the molecular weight is about 0.7 MDa and in yet another embodiment, the molecular weight is about 1.7 MDa. In some embodiments, the molecular weight is about 2.7 MDa.

At least a portion of the spherical forms as described herein is cross-linked. The term “cross-linked” is intended to refer to two or more polymer chains of hyaluronic acid which have been covalently bonded via a cross-linking agent. Such cross-linking is differentiated from intermolecular or intramolecular dehydration which results in lactone, anhydride, or ester formation within a single polymer chain or between two or more chains. Although, it is contemplated that intramolecular cross-linking may also occur in the spherical forms as described herein. The term “cross-linked” is also intended to refer to hyaluronic acid covalently linked to a BDDE derivative. In some embodiments, the term “cross-linked” also refers to covalently modified hyaluronic acid.

“Cross-linking agents” contain at least two reactive functional groups that create covalent bonds between two or more molecules. The cross-linking agents can be homobifunctional (i.e. have two reactive ends that are identical) or heterobifunctional (i.e. have two different reactive ends). The cross-linking agents to be used in the present disclosure should comprise complimentary functional groups to that of hyaluronic acid such that the cross-linking reaction can proceed. In one embodiment, the cross-linking does not form esterified hyaluronic acid. Suitable cross-linking agents include, by way of example only, butanediol diglycidyl ether (BDDE), divinyl sulfone (DVS), or 1-ethyl-3-(3-dimethylaminopropyl)carbodimide hydrochloride (EDC), or a combination thereof. In one embodiment, the cross-linking agent is BDDE. In one embodiment, the cross-linking agent is not a photocurable cross-linking agent.

As used herein, the term “BDDE derivative” refers to a form of BDDE wherein one or both epoxides of BDDE have reacted with hyaluronic acid. BDDE has the following chemical structure:

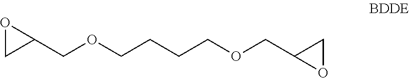 [SMILES: C(CCOCC1CO1)COCC1CO1]

One example of a BDDE derivative of hyaluronic acid is shown below.

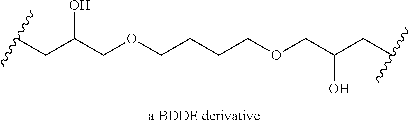 [SMILES: *CC(O)COCCCCOCC(O)C*]

The BDDE derivative of hyaluronic acid, as shown above, can be covalently bound to hyaluronic acid at both ends with both epoxides having been reacted. Additional BDDE derivatives of hyaluronic acid are contemplated herein. For example, certain BDDE derivatives of hyaluronic acid can be covalently bound at both ends between two separate hyaluronic acid polymers (i.e., cross-linked), while other BDDE derivatives can be covalently bound at both ends within a single hyaluronic acid polymer. Also contemplated are BDDE derivatives that are covalently bound at one or both ends to a hydroxyl group from one or more additional BDDE derivatives that are themselves covalently bound to hyaluronic acid.

Also contemplated are BDDE derivatives that that are covalently bound to hyaluronic acid at just one end. For example, one of the epoxide rings can be opened by covalent attachment to a single stretch of a hyaluronic acid polymer while the other epoxide ring can remain closed (i.e., unreacted). It is further contemplated that, within the cross-linked hyaluronic acid compositions, the concentration of such BDDE derivatives with an unreacted epoxide is sufficiently low so as not to affect the biocompatibility of spherical forms prepared from such compositions. Further contemplated is a BDDE derivative in which one of the epoxide rings has been opened by covalent attachment to a single stretch of hyaluronic acid polymer while the other epoxide ring has been opened by hydrolysis. However, it is contemplated that the cross-linked hyaluronic acid compositions comprise at least about 2 mole % BDDE (with respect to the disaccharide monomer) which is covalently bound at both ends between two separate hyaluronic acid polymers.

As used herein, the term “binder” refers to a naturally occurring or synthetic substance which provides uniform consistency and/or cohesion in the composition comprising the cross-linked hyaluronic acid, which when extruded, forms a spherical forms. In one embodiment, the binder is noncross-linked hyaluronic acid. In another embodiment, the binder is selected from a group consisting of sugars and polysaccharides such as sucrose, maltose, chondroitin sulfate, dermatan sulfate, heparin, chitosan, cellulose, gelatin, collagen, acacia, starch, PVP (polyvinyl pyrrolidone), HPC (hydroxypropyl cellulose), HPMC (hydroxypropyl methylcellulose), PEG, PLGA (poly(lactic-co-glycolic acid), carboxy methyl cellulose, ethylcellulose, gelatin polyethylene oxide, dextrin, magnesium aluminum silicate, polymethacrylates, and the like.

As used herein, the term “skin” refers to the three layers: the epidermis, the dermis, and the hypodermis or the deeper subcutaneous tissue.

As used herein, the term “spherical form” refers to sphere, rod, oval, cylinder forms of a material. Spherical forms are further defined elsewhere herein. The spherical form as described herein can have a variety of shapes in the cross-section, which are discussed below.

The spherical forms may be produced from forms having other shapes, for example, from thread forms, for example, from fragments of thread forms. Such thread forms can include into a variety of shapes. The term “substantially cylindrical” refers to a thread form wherein the cross-section is round. The term “substantially” as used to refer to shapes of the thread forms means that at least 50% of the thread form has the approximate shape described.

The term “percent moisture” is intended to refer to the total percent of water by weight. In one embodiment, the percent moisture of the spherical form is about 30% or less, or alternatively, about 15% or less, or alternatively, about 10% or less. This can typically be measured by Karl Fisher titration.

The term “therapeutic agent” can include one or more therapeutic agents. In still other of the above embodiments, the therapeutic agent is an anesthetic, including but not limited to, lidocaine, xylocaine, novocaine, benzocaine, prilocaine, ripivacaine, propofol, or combinations thereof. In still other of the above embodiments, the therapeutic agent includes, but is not limited to, epinephrine, ephedrine, aminophylline, theophylline or combinations thereof. In still other of the above embodiments, the therapeutic agent is botulism toxin. In still other of the above embodiments, the therapeutic agent is laminin-511. In still other of the above embodiments, the therapeutic agent is glucosamine, which can be used, for example, in the treatment of regenerative joint disease. In still other of the above embodiments, the therapeutic agent is an antioxidant, including but not limited to, vitamin E or all-trans retinoic acid such as retinol. In still other of the above embodiments, the therapeutic agent includes stem cells. In still other of the above embodiments, the therapeutic agent is insulin, a growth factor such as, for example, NGF (nerve growth factor), BDNF (brain-derived neurotrophic factor), PDGF (platelet-derived growth factor) or Purmorphamine Deferoxamine NGF (nerve growth factor), dexamethasone, ascorbic acid, 5-azacytidine, 4,6-disubstituted pyrrolopyrimidine, cardiogenols, cDNA, DNA, RNAi, BMP-4 (bone morphogenetic protein-4), BMP-2 (bone morphogenetic protein-2), an antibiotic agent such as, for example, β lactams, quinolones including fluoroquinolones, aminoglycosides or macrolides, an anti-fibrotic agent, including but not limited to, hepatocyte growth factor or Pirfenidone, an anti-scarring agent, such as, for example, anti-TGF-b2 monoclonal antibody (rhAnti-TGF-b2 mAb), a peptide such as, for example, GHK copper binding peptide, a tissue regeneration agent, a steroid, fibronectin, a cytokine, an analgesic such as, for example, Tapentadol HCl, opiates, (e.g., morphine, codone, oxycodone, etc.) an antiseptic, alpha-beta or gamma-interferon, EPO, glucagons, calcitonin, heparin, interleukin-1, interleukin-2, filgrastim, a protein, HGH, luteinizing hormone, atrial natriuretic factor, Factor VIII, Factor IX, or a follicle-stimulating hormone.

The term “diagnostic agent” refers to an agent which is used as part of a diagnostic test (e.g., a fluorescent dye to be used for viewing the spherical forms in vivo). In one embodiment, the diagnostic agent is soluble TB (tuberculosis) protein.

The term “lubricity-enhancing agent” is intended to refer to a substance or solution which when contacted with the dry spherical forms, acts to lubricate the dry spherical forms. A lubricity-enhancing agent can comprise, for example, water and/or an alcohol, an aqueous buffer, and may further comprise additional agents such as polyethylene glycol, hyaluronic acid, and/or collagen.

The term “biodegradation impeding agent” is intended to refer to a biocompatible substance that slows or prevents the in vivo degradation of the spherical forms. For example, a biodegradation impeding agent can include hydrophobic agents (e.g., lipids) or sacrificial biodegradation agents (e.g., sugars).

The term “firm” is intended to refer to a cohesive material that maintains its form in an unconstrained environment (i.e. as opposed to a flowable/amorphous material) and demonstrates some degree of structural integrity under compression. A gelatin cube is an example of a firm gel.

The term “aqueous gel composition” or “gel composition” or “gel mixture” is intended to refer to an aqueous composition comprising water, hyaluronic acid, and a cross-linking agent and/or cross-linked hyaluronic acid. In some embodiments, the composition may further comprise a buffer such that that the pH of the solution changes very little with the addition of components of the composition. In these embodiments, the composition is referred to as an aqueous buffered gel composition. The pH of the buffered gel composition is typically from about 7 to about 13. In certain embodiments the pH is about 7. In certain embodiments, the pH is higher at about 9 or about 10. In some embodiments, the pH can be adjusted by adding an appropriate amount of a suitable base, such as Na₂CO₃ or NaOH. In some embodiments, the aqueous gel buffered composition comprises phosphate buffered saline. In some embodiments, the aqueous gel buffered composition comprises tris(hydroxymethyl)aminomethane (Tris), which has the formula (HOCH₂)₃CNH₂. In some embodiments, additional solutes are added to adjust the osmolarity and ion concentrations, such as sodium chloride, calcium chloride, and/or potassium chloride.

The term “buffer” is intended to refer to a solution that stabilizes pH, wherein the solution comprises a mixture of a weak acid and its conjugate base or a weak base and its conjugate acid. Buffer solutions include, but are not limited to, 2-amino-2-methyl-1,3-propanediol, 2-amino-2-methyl-1-propanol, L-(+)-tartaric acid, D-(−)-tartaric acid, ACES, ADA, acetic acid, ammonium acetate, ammonium bicarbonate, ammonium citrate, ammonium formate, ammonium oxalate, ammonium phosphate, ammonium sodium phosphate, ammonium sulfate, ammonium tartrate, BES, BICINE, BIS-TRIS, bicarbonate, boric acid, CAPS, CHES, calcium acetate, calcium carbonate, calcium citrate, citrate, citric acid, diethanolamine, EPP, ethylenediaminetetraacetic acid disodium salt, formic acid solution, Gly-Gly-Gly, Gly-Gly, glycine, HEPES, imidazole, lithium acetate, lithium citrate, MES, MOPS, magnesium acetate, magnesium citrate, magnesium formate, magnesium phosphate, oxalic acid, PIPES, phosphate buffered saline, piperazine potassium D-tartrate, potassium acetate, potassium bicarbonate, potassium carbonate, potassium chloride, potassium citrate, potassium formate, potassium oxalate, potassium phosphate, potassium phthalate, potassium sodium tartrate, potassium tetraborate, potassium tetraoxalate dehydrate, propionic acid solution, STE buffer solution, sodium 5,5-diethylbarbiturate, sodium acetate, sodium bicarbonate, sodium bitartrate monohydrate, sodium carbonate, sodium citrate, sodium chloride, sodium formate, sodium oxalate, sodium phosphate, sodium pyrophosphate, sodium tartrate, sodium tetraborate, TAPS, TES, TNT, TRIS-glycine, TRIS-acetate, TRIS buffered saline, TRIS-HCl, TRIS phosphate-EDTA, tricine, triethanolamine, triethylamine, triethylammonium acetate, triethylammonium phosphate, trimethylammonium acetate, trimethylammonium phosphate, Trizma® acetate, Trizma® base, Trizma® carbonate, Trizma® hydrochloride or Trizma® maleate.

The term “aqueous solvent” is intended to refer to a non-toxic, non-immunogenic aqueous composition. The aqueous solvent can be water and/or an alcohol, and may further comprise buffers, salts (e.g., CaCl₂) and other such non-reactive solutes.

### 2. Spherical Forms and Methods of Preparing Spherical Forms

Spherical forms have been developed and methods of their preparation are described herein. The spherical forms contemplated herein comprising hyaluronic acid or a salt thereof, wherein at least a portion of the hyaluronic acid is cross-linked with BDDE. In another embodiment, the forms comprise noncross-linked hyaluronic acid or a salt thereof.

### Preparation of Cross-Linked Hyaluronic Acid

Spherical forms comprising cross-linked hyaluronic acid have been prepared according to methods described herein having increased ratios of cross-linking agent (e.g., BDDE derivatives) relative to the repeating disaccharide unit of the hyaluronic acid.

Generally, hyaluronic acid (HA) used herein has a molecular weight of from about 0.5 MDa (mega Dalton) to about 3.0 MDa. In some embodiments, the molecular weight is from about 0.6 MDa to about 2.6 MDa, and in yet another embodiment the molecular weight is from about 1.4 MDa to about 1.7 MDa. In some embodiments, the molecular weight is about 0.7 MDa, and in yet another embodiment the molecular weight is about 1.7 MDa. In some embodiments, the molecular weight is about 2.7 MDa.

In one aspect, there are provided compositions comprising cross-linked hyaluronic acid formed under aqueous conditions. In certain embodiments, such aqueous compositions form gels. In certain embodiments, the hyaluronic acid is hydrated for between about one minute and about 60 minutes prior to cross-linking. In other embodiments, the hyaluronic acid is hydrated for between about one hour and about 12 hours prior to cross-linking. In certain embodiments, the hyaluronic acid is hydrated for about one hour and in yet another embodiment the hyaluronic acid is allowed to hydrate for about two hours prior to cross-linking. In certain embodiments, the hyaluronic acid is hydrated for about three hours and in yet another embodiment the hyaluronic acid is allowed to hydrate for four hours prior to cross-linking.

Prior to addition of the HA, the aqueous solution is adjusted to the desired pH. In one embodiment, the aqueous solution has a pH>about 7. In certain embodiments, the solution has a pH of about 9, or about 10, or about 11, or about 12 or about 13, or greater than 13. Typically, the solution comprises water and can optionally comprise phosphate buffered saline (PBS) or tris(hydroxymethyl)aminomethane (Tris) buffer. The buffer can be selected based on the desired pH of the composition. For example, PBS can be used for compositions at a pH of about 7, whereas Tris can be used for compositions having a higher pH of about 9 or about 10. In some embodiments, the pH is from between about 9 and about 13. In some embodiments, the pH is at least about 13. In some embodiments, the pH is adjusted with the appropriate amount of a suitable base, such as Na₂CO₃ or NaOH to reach the desired pH. In some embodiments, the concentration of base is from about 0.00001 M to about 0.5 M. In some embodiments, the concentration of base is from about 0.1 M to about 0.25 M. In some embodiments, the concentration of base is about 0.2 M.

The concentration of hyaluronic acid used during the cross-linking contributes to the quality of the compositions comprising cross-linked hyaluronic acid. For example, the gels become increasingly firm when the concentration of hyaluronic acid used during cross-linking is at least about 5%. Further, it has been found that the gel swelling ratio in water can be increased by decreasing the concentration of hyaluronic acid used during the cross-linking. In one embodiment, the composition during the cross-linking comprises from about 1 weight % to about 25 weight % hyaluronic acid, before cross-linking. In another embodiment, the composition during the cross-linking comprises about 14 weight % hyaluronic acid, before cross-linking. In another embodiment, the composition during the cross-linking comprises about 12 weight % hyaluronic acid, before cross-linking. In another embodiment, the composition during the cross-linking comprises about 8 weight % hyaluronic acid, before cross-linking. In another embodiment, the composition during the cross-linking comprises about 5 weight % hyaluronic acid, before cross-linking.

In an alternative embodiment, the cross-linking is performed neat, i.e., without a solvent. Therefore, in certain embodiments, neat BDDE is contacted with dry hyaluronic acid to provide the cross-linked hyaluronic acid. The composition can then be hydrated with the desired amount of aqueous medium to provide the gel composition.

Compositions comprising cross-linked hyaluronic acid are formed when hyaluronic acid is contacted with a cross-linking agent. The cross-linking agent to be used in the present disclosure should comprise complimentary functional groups to that of hyaluronic acid such that the cross-linking reaction can proceed. The cross-linking agent can be homobifunctional or heterobifunctional. It is contemplated that the percent hydration of the spherical forms may be at least partially controlled by the type of cross-linking agent employed. For example, if the cross-linking leaves the carboxyl groups of the hyaluronic acid unfunctionalized, the percent hydration of the spherical forms may be higher than esterified hyaluronic acid. Suitable cross-linking agents include, but are not limited to, butanediol diglycidyl ether (BDDE), divinyl sulfone (DVS), and 1-ethyl-3-(3-dimethylaminopropyl)carbodiimide hydrochloride (EDC), or a combination thereof. In some aspects, there is provided a composition comprising substantially cross-linked hyaluronic acid, wherein hyaluronic acid is cross-linked or covalently modified with a BDDE derivative.

The amount of BDDE used is sufficient to produce a composition comprising at least 15 mole % of a BDDE derivative relative to the repeating disaccharide unit of the hyaluronic acid. In one embodiment, the composition comprises from about 15 mole % to about 25% mole percent, or about 17 mole % to about 20 mole % of the BDDE derivative, or about 18 mole % to about 19 mole %. In one embodiment, the composition comprises 18.75% of the BDDE derivative.

The amount of BDDE selected will be sufficient enough to provide a firm composition. For example, the gels become increasingly firm when the concentration of BDDE used during cross-linking is at least about 10 weight % relative to the weight of the hyaluronic acid. The amount of BDDE used during cross-linking, upon formation of the composition comprising cross-linked hyaluronic acid, may also be expressed as a weight % relative to the weight of the hyaluronic acid used during cross-linking. In one embodiment, between 25 weight % and 100 weight % BDDE is used relative to the weight of hyaluronic acid. In another embodiment, at least 30% BDDE is used relative to the weight of hyaluronic acid. In another embodiment, about 30% BDDE is used relative to the weight of hyaluronic acid. In another embodiment, at least 40% BDDE is used relative to the weight of hyaluronic acid. In another embodiment, about 40% BDDE is used relative to the weight of hyaluronic acid. In another embodiment, at least 50% BDDE is used relative to the weight of hyaluronic acid. In another embodiment, about 50% BDDE is used relative to the weight of hyaluronic acid. In another embodiment, between about 50% BDDE and about 75% BDDE is used relative to the weight of hyaluronic acid. In another embodiment, between about 75% BDDE and about 100% BDDE is used relative to the weight of hyaluronic acid.

### Washing, Drying, and Formulating Cross-Linked Hyaluronic Acid

After the cross-linked hyaluronic acid has been prepared, any excess cross-linking agent can be washed away. Water rinsing alone is typically insufficient to remove all excess cross-linking agent. Water rinsing can also be followed with or replaced by rinsing with a buffer and/or alcohol solvent, such as ethanol to remove the unreacted BDDE. It is contemplated that multiple washings may be necessary to remove all or substantially all of the excess cross-linking agent. It is further contemplated that the gels may be cut into smaller pieces or extruded from a syringe to improve the efficiency of the washing steps.

In some embodiments, the hydrated or washed gel pieces have been sized. The sizing be accomplished by loading the gel into a syringe and extruding through a delivery device (typically a 20 gauge (G) blunt needle) or through a screen (e.g., 355 μm screen). More than one or even a series of sizing steps can be performed using the same or a different, typically a smaller, gauge needle or screen than the previous sizing step. For example, the gel can be first extruded through a 20G needle once or twice, and then optionally extruded through a 23G or a 25G needle one or more times. In some embodiments, the more sizing steps implemented, the smoother the resultant spherical forms. Such results would be beneficial as a smoother spherical forms would ease delivery through the skin as the smoother spherical forms would exhibit less drag. It is further contemplated that multiple sizing steps may occur. For example, the first sizing step would result in a thread and the second sizing step would result in a spherical form.

In one embodiment, the compositions comprising cross-linked hyaluronic acid, as described above, are substantially dried (e.g., dehydrated) before being further combined with binder. In one embodiment, the aqueous gel compositions comprising cross-linked hyaluronic acid, as described above, are substantially dried (e.g., dehydrated) before being further combined with noncross-linked hyaluronic acid. In some embodiments, drying is accomplished by air-drying or first decanting the solvent before air drying at ambient or elevated temperatures. In one embodiment, drying is accomplished by lyophilization. In one embodiment, drying is partial.

In one embodiment, the compositions comprising cross-linked hyaluronic acid, as described above, are isolated via precipitation from a suitable solvent, such as ethanol, before being further combined with binder. The precipitation can be implemented multiple, i.e., more than one, time. In some embodiments, the particle size of the gel isolated via precipitation are substantially the same size as, or smaller than, the particles of the lyophilized gel. In certain embodiments, the spherical forms are made from the gel isolated via precipitation have a small dried spherical forms diameter (e.g., about 0.014″-0.015″), yet exhibit a faster rate of swelling, enhanced softness and larger swell diameter.

In one embodiment, the aqueous gel composition comprising cross-linked hyaluronic acid is dehydrated to remove about 25% of the water content by weight. In one embodiment, the aqueous gel composition comprising cross-linked hyaluronic acid is dehydrated to remove about 50% of the water content by weight. In one embodiment, the aqueous gel composition comprising cross-linked hyaluronic acid is dehydrated to remove about 75% of the water content by weight. In one embodiment, the aqueous gel composition comprising cross-linked hyaluronic acid is dehydrated to remove about 90% of the water content by weight.

It is contemplated that the concentration of total HA solids, including cross-linked hyaluronic acid and noncross-linked binder, within the aqueous gel compositions prior to extrusion, improves certain properties of the spherical forms as described herein such as smoothness and ease of handling either before or after extrusion.

The dried cross-linked hyaluronic acid composition, as described above, can be combined with water to form a substantially cross-linked hyaluronic acid gel, which can then optionally be formulated with a binder (e.g., noncross-linked hyaluronic acid) and/or additive (e.g., a salt, excipient, lidocaine, or the like). The resulting formulated gel composition comprising cross-linked hyaluronic acid, and optional binder such as noncross-linked hyaluronic acid, can then be extruded into a wet spherical form, which can then be dried.

In one embodiment, the cross-linked hyaluronic acid is present in the composition, before spherical forms drying and optionally with a binder such as noncross-linked hyaluronic acid, in an amount of from about 5 weight % to about 20 weight % based on the total weight of the composition. In still another embodiment, the cross-linked hyaluronic acid is present in the composition, before spherical forms drying and optionally with a binder such as noncross-linked hyaluronic acid, in an amount of from about 5 weight % to about 12 weight % or about 8 weight % to about 10 weight % based on the total weight of the composition, excluding moisture.

In one embodiment, there is provided a composition comprising the substantially cross-linked hyaluronic acid of any of the above embodiments, that has been dried and rehydrated, and further comprises a binder. In one embodiment, the binder is noncross-linked hyaluronic acid. In one embodiment, the binder, such as noncross-linked hyaluronic acid, is provided as an aqueous solution. In one embodiment, the binder, such as noncross-linked hyaluronic acid, is provided as an aqueous solution further comprising a salt, such as CaCl₂. In one embodiment, the binder, such as noncross-linked hyaluronic acid, is provided as an aqueous solution further comprising 1 mM CaCl₂, 2.5 mM CaCl₂, 10 mM CaCl₂, or greater than 10 mM CaCl₂.

It is contemplated that the quality of the dry spherical forms as described herein may be dependent, at least partially, upon the quantity of the total hyaluronic acid solids used to make the wet spherical forms compositions, as described above. The “hyaluronic acid solids” include any combination of substantially cross-linked hyaluronic acid and/or noncross-linked hyaluronic acid (i.e., binder). In some embodiments, a minimum quantity of hyaluronic acid solids contributes to the quality of the dry spherical forms.

In one embodiment, the wet spherical forms or precursor shape (e.g. thread) comprises from about 2% to about 50% hyaluronic acid solids. In one embodiment, the wet spherical forms comprise from about 2% to about 40% hyaluronic acid solids. In one embodiment, the wet spherical forms comprise from about 2% to about 20% hyaluronic acid solids. In one embodiment, the wet spherical form comprises at least 7% hyaluronic acid solids. In one embodiment, the wet spherical form comprises at least 10% hyaluronic acid solids. In one embodiment, the wet spherical form comprises at least 12% hyaluronic acid solids. In one embodiment, the wet spherical form comprises at least 15% hyaluronic acid solids. In one embodiment, the wet spherical form comprises at least 18% hyaluronic acid solids. In one embodiment, the wet spherical form comprises at least 20% hyaluronic acid solids. In one embodiment, the wet spherical form comprises at least 25% hyaluronic acid solids.

As described above, the hyaluronic acid solids that are used to make the wet and dry spherical forms or precursor shape (e.g. thread) as described herein can include any combination of cross-linked hyaluronic acid and/or noncross-linked hyaluronic acid (i.e., binder). Adjustments in the quantity and/or ratio of cross-linked hyaluronic acid and/or noncross-linked hyaluronic acids can improve certain properties of the spherical forms (e.g., the swelling ratio by which the dry spherical forms absorb water, the smoothness of the dry spherical forms, the resistance of the dry spherical forms to in vitro enzymatic digestion by hyaluronidase, and/or an increased in vivo half-life).

In one embodiment, the cross-linked hyaluronic acid is present in compositions used to make spherical forms in an amount of from about 1 weight % to about 25 weight % based on the total weight of the composition. In another embodiment, the cross-linked hyaluronic acid is present in an amount of from about 2 weight % to about 15 weight % based on the total weight of the composition. In another embodiment, the cross-linked hyaluronic acid is present in an about 14 weight %. In another embodiment, the cross-linked hyaluronic acid is present in an about 12 weight %. In another embodiment, the cross-linked hyaluronic acid is present in an about 8 weight %. In another embodiment, the cross-linked hyaluronic acid is present in an about 5 weight %.

In one embodiment, the noncross-linked hyaluronic acid is present in compositions used to make spherical forms in an amount of from about 1 weight % to about 20 weight % based on the total weight of the composition. In another embodiment, the noncross-linked hyaluronic acid is present in an amount of from about 1 weight % to about 8 weight % based on the total weight of the composition. In another embodiment, the noncross-linked hyaluronic acid is present in an about 5 weight %. In another embodiment, the noncross-linked hyaluronic acid is present in an about 3 weight %. In another embodiment, the noncross-linked hyaluronic acid is present in an about 2 weight %.

In one embodiment, the compositions used to make spherical forms comprise from about 5 weight % to about 15 weight % cross-linked hyaluronic acid and from about 2 weight % to about 8 weight % noncross-linked hyaluronic acid. In one embodiment, the composition comprises about 12 weight % cross-linked hyaluronic acid and about 3 weight % noncross-linked hyaluronic acid. In one embodiment, the composition comprises about 8 weight % cross-linked hyaluronic acid and about 2 weight % noncross-linked hyaluronic acid. In one embodiment, the composition comprises about 5 weight % cross-linked hyaluronic acid and about 5 weight % noncross-linked hyaluronic acid. Compositions used to make spherical forms can be made with higher or lower concentrations of HA and cross-linked HA; the above three compositions are given as examples only.

### Deaerating, Extruding, and Drying Gels

In some embodiments, the aqueous gel composition comprising cross-linked and noncross-linked hyaluronic acid is deaerated (i.e., degassed), prior to extrusion or shape formation to minimize air bubbles after extrusion. The degassing can also be done by using a syringe.

In some embodiments, the compositions used to make the spherical forms or precursor shape (i.e., thread) are deaerated at least once. In some embodiments, the compositions used to make the spherical forms are deaerated more than once. In some embodiments, the compositions used to make the spherical forms are deaerated between two and ten times. In some embodiments, the compositions used to make the spherical forms are deaerated twice. In some embodiments, the compositions used to make the spherical forms are deaerated three times, four, five, six, seven, eight, nine, or ten times. In some embodiments, the compositions used to make the spherical forms are deaerated at least ten times.

To form the spherical forms, for example, the aqueous gel composition comprising cross-linked and noncross-linked hyaluronic acid is typically extruded onto a substrate. The substrate can provide the desired shape or form. The composition is extruded using a pressurized syringe affixed to a nozzle. The nozzle can have various geometries, such as various lengths, internal diameters and shapes. In one embodiment, the substrate provides a precursor shape, such as a thread. The thread is then cut into pieces of any desired length and placed into a shaker. The pieces are then shaken by the shaker until the desired spherical form is obtained.

In another embodiment, spherical forms may be formed by cutting the aqueous gel composition comprising cross-linked and noncross-linked hyaluronic acid as the gel is being extruded from a pressurized syringe to form the precursopr shape, for example, a thread form. The thread form may be cut into pieces and the pieces can then be placed into a shaker and shaken until the desired spherical form is obtained.

When the thread forms are made via extrusion, the nozzle may be circular or non-circular in shape, for example, a flattened shape or a “D” shape. The syringe nozzle may be anywhere from about a 15 gauge to a 25 gauge syringe nozzle. In some embodiments, the syringe nozzle is a 15 gauge nozzle, whereas in other embodiments the syringe nozzle is a 16 gauge nozzle. In some embodiments, the syringe nozzle is a 17 gauge nozzle, whereas in other embodiments the syringe nozzle is a 18 gauge nozzle. In some embodiments, the syringe nozzle is a 19 gauge nozzle, whereas in other embodiments the syringe nozzle is a 20 gauge nozzle. In some embodiments, the syringe nozzle is a 21 gauge nozzle, whereas in other embodiments the syringe nozzle is a 22 gauge nozzle. Typically, the pressure employed is from about 10 to about 2000 psi or from about 20 to about 240 psi. The pressure requirements are dictated by the nozzle geometry and other attributes such as consistency of the composition and desired flow rate. The pressure can be applied pneumatically, for example using ambient air or nitrogen, hydraulically, or mechanically. The speed at which the gel is extruded takes into consideration minimization of air bubbles in the forms and maximization of a consistent uniform shape. Air bubbles can reduce the structural integrity of the spherical forms by causing weak spots.

Pneumatic pressure and plate speed are not fixed but are instead adjusted and monitored in-process so that the gel is extruded in a continuous, linear manner. For a given lot of threads, the pneumatic pressure is first increased to the point of consistent but controllable gel flow. The plate speed is then continuously fine-tuned so that threads are extruded in a uniform linear manner. If the plate speed is too slow zig-zagged threads may result; too fast and threads may stretch leading to necking and/or breakage.

Various substrates are contemplated for use by methods as described herein. Substrates include by hydrophilic and hydrophobic substrates and may be selected from, but are not limited to, polytetrafluoroethylene (PTFE), expanded PTFE, nylon, polyethylene terephthalate (PET), polystyrene, silicon, polyurethane, and activated cellulose.

The substrate employed, along with the viscosity of the gel composition, dictates the general shape of the thread. For example, if the gel and the substrate have an equilibrium contact angle of less than 90 degrees, it is contemplated that the thread formed could be substantially ribbon-shaped. Further, if the gel and the substrate have an equilibrium contact angle of about 90 degrees, the thread formed could be substantially D-shaped. Still further, if the gel and the substrate have an equilibrium contact angle of greater than 90 degrees, then the thread formed could be substantially round. For example, a 10% 1.5 MDa gel will have a substantially circular cross-section (e.g., about 80% of a circle) when extruded on PTFE, while a 5% 1.5 MDa gel will form a flat ribbon when extruded on PTFE.

Alternative to pressurized extrusion, the gel composition can be rolled out into an elongated cylinder and/or cut into elongated strips before drying.

In further embodiment, spherical forms may be formed, for example, by extruding the aqueous gel composition comprising cross-linked and noncross-linked hyaluronic acid into spherical molds.

The wet spherical forms are then dried to form a dry spherical forms. Drying may be conducted under static conditions or, alternatively, with the assistance of a dynamic air flow (i.e., within a laminar flow hood). In some embodiments, yields of the spherical forms improve with static drying. As the spherical forms may lose some of their properties when exposed to heat in excess of water boiling temperature, it is preferred that the drying step be performed under ambient conditions.

The spherical forms are allowed to dry for anywhere from about 30 minutes to about 72 hours to form spherical forms having a diameter of from about 0.003″ to about 0.05″; or even an about 0.004″ to about 0.03″ diameter spherical forms will be sufficient. However, in some embodiments, the spherical forms are from about 0.003″ to about 0.03″, or from about 0.006″ to about 0.03″, or from about 0.008″ to about 0.03″, or from about 0.01″ to about 0.03″. In some embodiments, the size of the spherical forms is from about 0.010″ to about 0.020″ in diameter, or from about 0.020″ to about 0.025″ having 10%-30% by weight hydration. In some embodiments, the spherical forms can be dried for about 12 hours or about 24 hours. It is contemplated that the larger the molecular weight of HA employed or the more concentrated the HA in the composition, the longer the drying times that are required. Further, during the drying process, a non-thermal stimulus, such UV light, radiation, or a chemical initiator or a catalyst, may be employed to assist in the cross-linking reaction.

In some embodiments, after drying, the spherical forms are washed with an aqueous or non-aqueous solvent, a gas or a supercritical fluid. In some instances, this washing removes excess cross-linking agent. The washing can be accomplished by a variety of methods, such as submersion in an aqueous solvent or by using a concurrent flow system by placing the spherical forms in a trough at an incline and allowing an aqueous solvent to flow over the spherical forms.

In one embodiment, water is used to wash the spherical forms. In this embodiment, the water not only washes the spherical forms to remove excess cross-linking agent, it also rehydrates the spherical forms into a hydrated elastomeric state. In one embodiment, an antioxidant solution is used to wash the spherical forms. For example, in one embodiment, a buffer solution comprising ascorbic acid, vitamin E and/or sodium phosphate is used to wash the spherical forms. In one embodiment, a buffer solution comprising about 1 mM, or about 10 mM or about 100 mM, or about 1 M ascorbic acid is used to wash the spherical forms.

The percent hydration of hyaluronic acid can range from about 1% to greater than about 1000% based on the total weight. The percent hydration of the spherical forms of the present disclosure can be controlled by adjusting the percent hyaluronic acid in the gel and/or controlling the amount and type of cross-linking agent added. In some embodiments, the spherical forms have no more than about 30% percent, or no more than 15%, or no more than 10% by weight hydration based on the total weight. The percent hydration will be determined by the environment to which the spherical forms are subjected to during or after the drying process.

It is contemplated that the half-life of the hyaluronic acid spherical forms in vivo can be controlled by controlling the thickness of the spherical forms, the density, the degree of cross-linking, the molecular weight of the hyaluronic acid and the degree of hydration, which can then be further controlled by adjusting the amounts of hyaluronic acid and cross-linking agent both individually and relatively. It is contemplated that the spherical forms disclosed herein can have an enhanced half-life in vivo of from about 1 month to up to about 12 months as compared to less than 1 day for natural hyaluronic acid. In certain embodiments, it is contemplated that the spherical forms described herein have an in vivo half-life of at least 1 month. In certain embodiments, it is contemplated that the spherical form described herein have an in vivo half-life of at least 2 months. In certain embodiments, it is contemplated that the spherical forms described herein have an in vivo half-life of at least 3 months. In certain embodiments, it is contemplated that the spherical forms described herein have an in vivo half-life of at least 4 months. In certain embodiments, it is contemplated that the spherical forms described herein have an in vivo half-life of at least 6 months. In certain embodiments, it is contemplated that the spherical forms described herein have an in vivo half-life of at least 8 months. In certain embodiments, it is contemplated that the spherical forms described herein have an in vivo half-life of at least 10 months. In certain embodiments, it is contemplated that the spherical forms described herein have an in vivo half-life of at least 12 months. In certain embodiments, it is contemplated that the spherical forms described herein have an in vivo half-life of at least 14 months. In certain embodiments, it is contemplated that the spherical forms described herein have an in vivo half-life of at least 16 months. In certain embodiments, it is contemplated that the spherical forms described herein have an in vivo half-life of at least 18 months.

It is contemplated that the spherical forms described herein can be sterilized using typical sterilization methods known in the art, such as autoclave, ethyleneoxide, electron beam (e-beam), supercritical CO₂ (with peroxide), etc. For example, the spherical forms as described herein can be sterilized using electron beam (e-beam) sterilization methods. In some embodiments, the spherical forms are first washed in a buffer solution at high pH (i.e., pH 9 or pH 10). In some embodiments, the wash solutions further comprise ethanol, ascorbic acid, vitamin E and/or sodium phosphate.

After the spherical forms are rehydrated, they is allowed to dry again under ambient conditions for from anywhere from 30 minutes to about 72 hours. Upon drying, the spherical forms, in some embodiments, become cured which may to provide a more uniform surface of the spherical forms.

This washing hydration/dehydration step can be performed multiple times to allow excess unreacted reagent to be washed from the spherical forms or to continue to improve the degree of cross-linking or covalent modification. This is an improvement over methods such as the use of organic solvents to remove excess BDDE.

### 3. Nomenclature

Spherical forms as described herein are prepared from compositions comprising substantially cross-linked hyaluronic acid. In certain embodiments, spherical forms are prepared from compositions comprising substantially cross-linked hyaluronic acid, and further comprising a binder such as noncross-linked hyaluronic acid. Spherical forms can be described according to the following nomenclature AA/BB@XX/YY, wherein AA/BB describes the initially formed composition comprising substantially cross-linked hyaluronic acid and XX/YY describes the composition with a binder, such as noncross-linked hyaluronic acid.

For example, spherical forms referred to as “10/40@15/20” refers to a composition of substantially cross-linked hyaluronic acid, wherein the cross-linking reaction is performed with 10 weight % hyaluronic acid relative to the weight of the solution (e.g., an aqueous solution), using 40 weight % cross-linker (e.g., BDDE) relative to the weight of the hyaluronic acid.

As referred to herein, AA of AA/BB@XX/YY is at least 2%. In some embodiments, AA is at least 5%. In some embodiments, AA is about 8%. In some embodiments, AA is about 10%. In some embodiments, AA is at least 10%. In some embodiments, AA is about 12%. In some embodiments, AA is at least 15%.

In some embodiments, BB of AA/BB@XX/YY is at least 10%. In some embodiments, BB is at least 20%. In some embodiments, BB is at least 30%. In some embodiments, BB is about 30%. In some embodiments, is at least 40%. In some embodiments, BB is about 40%. In some embodiments, BB is at least 50%. In some embodiments, BB is about 50%.

In some embodiments, AA/BB of AA/BB@XX/YY is about 8/10. In some embodiments, AA/BB is at least or about or exactly 8/20. In some embodiments, AA/BB is about 8/30. In some embodiments, AA/BB is about 8/40. In some embodiments, AA/BB is about 8/50. In some embodiments, AA/BB is about 10/10. In some embodiments, AA/BB is about 10/20. In some embodiments, AA/BB is about 10/30. In some embodiments, AA/BB is about 10/40. In some embodiments, AA/BB is about 10/50.

In some embodiments, one or more binding agents, such as noncross-linked hyaluronic acid, are added to the compositions comprising cross-linked hyaluronic acid, and the resulting compositions are converted by additional methods described herein to provide novel spherical forms. In some embodiments, the compositions comprising cross-linked hyaluronic acid further comprise noncross-linked hyaluronic acid. The added noncross-linked hyaluronic acid is optionally referred to herein as a “binder.” The combination of cross-linked hyaluronic acid and noncross-linked hyaluronic acid, within the composition, is optionally referred to herein as “hyaluronic acid solids.” As referred to herein, the XX of AA/BB@XX/YY refers to the weight % of total hyaluronic acid solids relative to the weight of the composition, wherein the hyaluronic acid solids includes both the substantially cross-linked hyaluronic acid and any noncross-linked hyaluronic acid. As referred to herein, the YY of AA/BB@XX/YY refers to the weight % of noncross-linked hyaluronic acid relative to the weight of total hyaluronic acid solids.

In some embodiments, XX of AA/BB@XX/YY is at least 2%. In some embodiments, XX is at least 5%. In some embodiments, XX is at least 10%. In some embodiments, XX is about 10%. In some embodiments, XX is at least 15%. In some embodiments, XX is about 15%. In some embodiments, XX is at least 20%. In some embodiments, XX is about 20%. In some embodiments, XX is at least 25%. In some embodiments, XX is about 25%.

In some embodiments, YY of AA/BB@XX/YY is at least 5%. In some embodiments, YY is at least 10%. In some embodiments, YY is at least 20%. In some embodiments, YY is about 20%. In some embodiments, YY is at least 30%. In some embodiments, YY is about 30%. In some embodiments, YY is at least 40%. In some embodiments, YY is about 40%. In some embodiments, YY is at least 50%. In some embodiments, YY is about 50%.

In some embodiments, AA/BB@XX/YY is 8/10@2/5, 8/20@5/10, 8/30@10/20, 8/40@15/20, 8/50@20/30, 10/10@20/40, 10/20@25/50, 10/30@20/40, 10/40@10/50, 10/50@20/40, and the like.

In certain embodiments, disclosed herein are spherical forms comprising substantially cross-linked hyaluronic acid, wherein the hyaluronic acid is substantially cross-linked with at least about 15 mole % of a butanediol diglycidyl ether (BDDE) derivative relative to the repeating disaccharide unit of the hyaluronic acid, and at least about 5% noncross-linked hyaluronic acid relative to the weight of total hyaluronic acid solids, wherein the cross-linked hyaluronic acid is present in an amount of from about 60 weight % to about 90 weight % based on the total weight of the spherical forms excluding moisture and the noncross-linked hyaluronic acid is present in an amount of from about 10 weight % to about 40 weight % based on the total weight of the spherical forms excluding moisture.

### 4. Modification of the Spherical Forms

In addition to washing the spherical forms, it can also be further functionalized by adsorbing a sufficient amount of a member selected from the group consisting of a therapeutic agent, a diagnostic agent, a fibrogenesis-enhancing agent, a biodegradation impeding agent, a lubricity-enhancing agent and combinations thereof, optionally followed by re-drying the spherical form. Such therapeutic agents include antibacterials, anesthetics, dyes for viewing placement in vivo, and the like. In some embodiments, a dry or hydrated spherical form is coated to alter the properties with a bioabsorbable biopolymer, such as collagen, PEG, PLGA or a phase transfer Pluronic™ which can be introduced as a liquid and which solidifies in vivo.

In one embodiment, the spherical forms can be coated to modulate the rate at which the spherical forms are rehydrated. For example, the spherical forms can be coated with a hydrophobic layer, such as a lipid. The thickness of the lipid layer can then be adjusted to achieve the desired rate of rehydration. In another embodiment, the spherical forms can be coated with an aqueous composition of noncross-linked hyaluronic acid. This can be performed just prior to implantation of the spherical forms to act as a lubricant. It is also contemplated that this coating with noncross-linked hyaluronic acid may slow the rate of hydration of the spherical forms. In some embodiments, the spherical forms are coated, either totally or in part, with the gel composition to form a layered material. Woven constructs, whether single layer or 3D, can be coated in their entirety to create weaves or meshes with altered physical properties from that of a free-woven mesh.

### 5. Methods of Using the Cross-Linked Hyaluronic Acid Spherical Forms

The spherical forms, or similar structures described herein can be used, for example, to fill wrinkles, nose augmentation or reconstruction, lip augmentation or reconstruction, facial augmentation or reconstruction.

### Methods of Treating a Wrinkle

Tissue repair could prolong the “filler” effects of the spherical forms when used to treat or fill a wrinkle in vivo far beyond the half-life of the hyaluronic acid-based spherical forms as described herein.

In some embodiments, there is provided a method of treating a wrinkle in a patient in need thereof by 1) inserting (e.g. by a delivery device or means of delivery) a plurality of spherical forms as described herein into the dermis or subcutaneous space of the patient adjacent to or under the wrinkle; and 2) applying the plurality of spherical forms adjacent to or under the wrinkle thereby treating the wrinkle. These steps can be performed at least once and up to 6 times to treat each wrinkle. In certain embodiments, step 2 can be performed at least two or more times. Optionally and as necessary, the plurality of spherical forms are hydrated with water or saline, or by the fluids normally perfusing the surrounding tissue. Further, the remainder of the wrinkle can be filled with a biocompatible material such as a phase transfer Pluronic™ which can be introduced as a liquid and which solidifies in vivo. Alternatively, conventional hyaluronic acid gel can be introduced to fill the wrinkle. In either case, the formed web acts to maintain the biocompatible filler at the site of the wrinkle.

In some embodiments, a method of treating a wrinkle in a subject is provided. In some embodiments, the attending clinician may numb the treatment area according to procedures known in the art using a variety of anesthetics, including, but not limited to, topical lidocaine, ice or a block with lidocaine injection. The distal end of the delivery device may be inserted through the skin surface of the subject into the dermis adjacent to or within the wrinkle. In some embodiments, spherical forms are inserted into the subcutaneous space instead of the dermis. The delivery device then may traverse the dermis or subcutaneous space of the subject beneath the wrinkle.

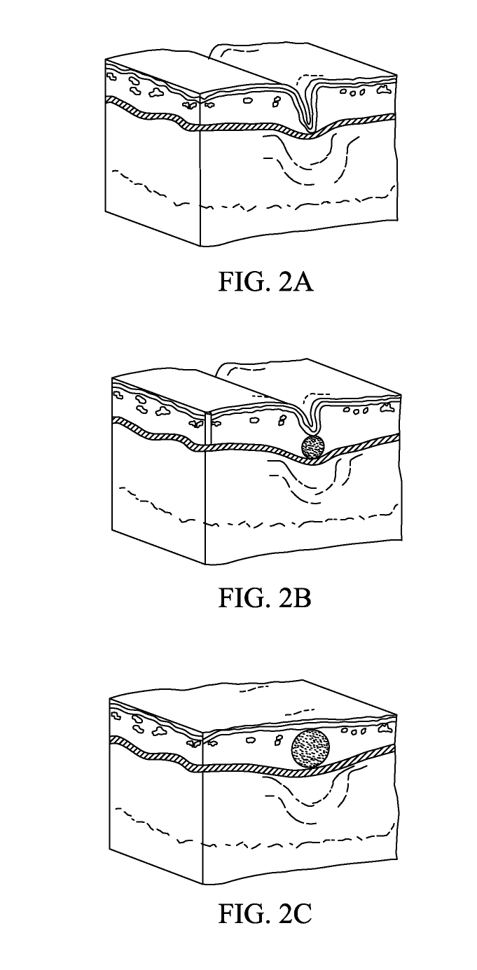

The method above may successfully treat wrinkles as shown in FIGS. 2A, 2B and 2C. A typical wrinkle is illustrated in FIG. 2A. FIG. 2B illustrates a spherical form implanted beneath a wrinkle, the form not yet hydrated. A plurality of spherical forms can be implanted adjacent one another along the length of a wrinkle. As the plurality of spherical forms are implanted beneath the wrinkle, they becomes fully hydrated and the surface appearance of the wrinkle is concurrently flattened as illustrated in FIG. 2C.

### Facial Contouring

The spherical forms as described herein are useful in facial contouring. What is meant by facial contouring is that a plurality of spherical forms can be applied to any area of the face, neck, or chest that the patient desires to have augmented, including, by way of example only, the lips, the nasolabial fold, and tear trough.

Lip augmentation is a commonly desired aesthetic procedure. Typically, the aesthetic goal is fuller, plumper lips. Available treatment options for lip augmentation include temporary fillers such as Restylane® and Juvederm®, permanent fillers such as ArteFill®, Radiesse® and Goretex® implants, as well as surgical procedures. Areas of enhancement can include the vermillion border (or white roll) for lip effacement and contouring and the wet-dry mucosal junction for increasing fullness. Other techniques include more diffuse infiltration of the orbicularis oris muscle.

Lip contouring and augmentation by temporary soft tissue augmentation products is a popular, low risk option due to the minimal invasiveness and temporary nature of the procedure. The major shortcomings of soft tissue augmentation products currently used in lip procedures are that it is (a) painful, (b) difficult to consistently and homogenously inject the gel into the desired location, and (c) the gel can migrate over the lifetime of the implant causing the aesthetic results to change.

In embodiments directed to facial contouring, the attending clinician may numb the treatment area according to procedures known in the art using a variety of anesthetics, including, but not limited to, topical lidocaine, ice, or a block with lidocaine injection. A plurality of spherical forms made of HA (hyaluronic acid) can be inserted by a delivery device into the lip. Using a delivery device, the plurality of spherical forms can be implanted with precision into the desired location. It is contemplated that when spherical forms are used for facial contouring, any number of spherical forms may be used depending on the desired effect and the size of the spherical forms.

It is contemplated that implanting the plurality of spherical forms in various planes may also be done in the treatment of wrinkles as described above.

### 6. Kits

Also proved herein is a kit of parts comprising spherical forms as described herein. In some embodiments, the kit comprises a plurality of spherical forms and a means for delivering or implanting the plurality of spherical forms to a patient. In one embodiment, the means for delivery to a patient is a delivery device or means of delivery. In another embodiment, the means for delivery to a patient is an air gun. The size (or diameter) of the delivery device may depend on the use of the spherical forms, and therefore, also be based on the cross-sectional area of the spherical form used. It is further contemplated that the outer diameter of the spherical forms may be larger than the outer diameter of the delivery device. Skin is quite pliable so by having a smaller diameter delivery device can allow the puncture size to be small even with the use of a larger diameter spherical forms.

Further, the size of the delivery device, will be dependent on its intended use and the size of the spherical form. It is contemplated that for use in facial contouring and/or wrinkle filling the spherical forms are from about 0.003″ to about 0.05″; or even an about 0.004″ to about 0.03″ diameter spherical forms will be sufficient. However, in some embodiments, the spherical forms are from about 0.003″ to about 0.03″, or from about 0.006″ to about 0.03″, or from about 0.008″ to about 0.03″, or from about 0.01″ to about 0.03″. In further embodiments, the size of the spherical forms are from about 0.010″ to about 0.020″ in diameter, or from about 0.020″ to about 0.025″ will be sufficient.

### EXAMPLES

The present disclosure is further defined by reference to the following examples. It will be apparent to those skilled in the art that many modifications, to spherical forms, threads and methods, may be practiced without departing from the scope of the current disclosure. The hyaluronic acid and cross-linking agents are available from commercial sources.

### Example 1

### Synthesis of a Cross-Linked Thread

Cross-linked hyaluronic acid threads can be made according to the following procedures. As noted above, after forming the threads, the threads can then be formed into spherical forms, e.g. spheres for use in tissue augmentation, in accordance with certain embodiments of the invention.

### Cross-Linking: Preparation of Cross-Linked Hyaluronic Acid Gel

Hyaluronic acid (HA) powder is hydrated in about 75% of a desired total volume of NaOH for about 30 minutes at about 50° C. in an appropriate container. The hydrated HA is then added to a syringe and mixed thoroughly (e.g., syringe-to-syringe about 20 times). Heating is continued for approximately 30 minutes.

Cross-linker (e.g., BDDE) is then dissolved in the remaining portion of the desired total volume of NaOH (i.e., about 25% of the desired total volume), added to the hydrated hyaluronic acid solution (dropwise or in one portion), mixed thoroughly (e.g., syringe-to-syringe about 20 times), heated for about 30 minutes, re-mixed (e.g., syringe-to-syringe about 20 times), and transferred to an appropriate container. Heating is then continued at about 50° C. for an additional 3 to 5 hours.

Various cross-linked gels were prepared with differing concentrations of components using the procedure described hereinabove.
HA molecular weight (MDa): e.g., 0.7, 1.7, 2.7.
HA hydration time: e.g., 0 minutes, 30 minutes, 1 hour, 2 hours, overnight.
Reaction pH: 9-13.4 using, e.g., 0.00001-0.25 M NaOH.
Cross-linking reaction time: e.g., 3-4.5 hours.
HA concentration (c)/0 w/w HA:aqueous NaOH): e.g., 5, 6, 7, 8, 9, 10, 11, 12.
BDDE concentration (% w/w BDDE:HA): e.g., 0.5, 2, 2.5, 5, 7.5, 8, 10, 20, 30, 40, 100.

### Rinsing/Optional Sizing

The cross-linked hyaluronic acid gel is rinsed to remove the sodium hydroxide and any unreacted BDDE. Water can also be removed, and the gels may be fragmented to facilitate the extrusion step and/or for rinsing efficiency. In such an instance, sizing is accomplished by cutting the gel into approximately 0.5 cm cubes. Rinsing is then achieved by rinsing with about 40 volumes of 10 mM sodium phosphate, at pH 6.0, three times for about 30 minutes each, rinsing with 100% ethanol six times for about 30 minutes, and then rinsing with water four times for about 30 minutes.

The swollen gel pieces can then be further sized into approximately 0.25 cm cubes, loaded into a syringe, and extruded through a 20 gauge (G) blunt needle. More than one sizing step can be performed using the same or a different, typically smaller, gauge needle (e.g., 20G, then two 25G sizing steps).

### Drying

The cross-linked hyaluronic acid gel is then diluted approximately 2:1 with water, loaded into a pan and dried. The drying is accomplished by air drying under ambient conditions or lyophilization. Alternatively, the gel is isolated via precipitation from ethanol. The gel is either partially dried to a desired final concentration or dried completely.

### Formulating

The completely dried cross-linked hyaluronic acid gel is formulated as an aqueous composition to the desired final HA concentration (e.g., 2.5%, 5%, 7.5%, 10%, 12.5%, 15%, 20%). The partially dried cross-linked hyaluronic acid gel can be used as an aqueous composition in the formulation without further treatment.

The aqueous composition of cross-linked hyaluronic acid gel can then be further formulated with a binder such as noncross-linked hyaluronic acid. In such a case, noncross-linked hyaluronic acid is hydrated (e.g., overnight at 4° C.) at the desired final HA concentration (e.g., 2.5%, 5%, 7.5%, 10%, 12.5%, 15%, 20%). The binder is mixed with the cross-linked hyaluronic acid gel. Typical binders include noncross-linked hyaluronic acid, salts (e.g., CaCl₂), excipients, Lidocaine, and the like.

The aqueous composition can comprise any aqueous medium, such as an acid, a base, a buffer or a salt. Buffers such as phosphate buffered saline can be used (e.g., 10 mM PBS at pH 7.4). Calcium chloride solutions can also be used (e.g., 1 mM, 2.5 mM, or 5 mM). Sodium hydroxide (NaOH) solutions may be used, (e.g., 0.1M, 0.2M, 0.3M, or 0.5M).

Compositions can be made with higher or lower concentrations of hyaluronic acid and cross-linked HA; the following three compositions are given as examples only.

In one composition, for example, the final extruded composition contained 12% (w/w) cross-linked hyaluronic acid and 3% (w/w) noncross-linked HA, wherein the cross-linked hyaluronic acid was derived from a cross-linking reaction with either 10% hyaluronic acid and 4% BDDE, or 8% hyaluronic acid and 3.2% BDDE.

In another composition, the final extruded composition contained 8% (w/w) cross-linked hyaluronic acid and 2% (w/w) noncross-linked HA, the cross-linked hyaluronic acid being derived from a cross-linking reaction with either 10% hyaluronic acid and 4% BDDE, or 8% hyaluronic acid and 3.2% BDDE.

In yet another composition, the final extruded composition contained 5% (w/w) cross-linked hyaluronic acid and 5% (w/w) noncross-linked HA, the cross-linked hyaluronic acid being derived from a cross-linking reaction with 10% hyaluronic acid and 4% BDDE.

### Extruding

The final gel formulations may be then extruded onto a suitable surface to yield wet threads. Various nozzle sizes are used depending on the final desired thickness (e.g., 20G, 19G, 18G, 17G, 16G).

The final gel formulations are transferred to a pressurized extruder (e.g., EFD Model XL1500 pneumatic dispense machine). The nozzle of the extruder can have a tip ranging from a 15 gauge to about 25 gauge. The syringe pressure may be between about 10 psi and about 2000 psi, depending on the viscosity of the final gel formulation. For very viscous gel formulations, a pressure multiplier can be used.

The wet threads was then formed by extruding the final gel formulation onto a substrate by an extruder to achieve the desired wet threads thickness. For example, to achieve a similar dried diameter, one can use a 20 gauge needle for 15% hyaluronic acid compositions, or a 19 gauge needle for 10% hyaluronic acid compositions.

### Drying

The wet threads can then be dried under ambient conditions to a percent hydration of less than about 30%, or less than about 15%, or less than about 10%, thus providing a dry threads. Optionally, the threads can be allowed to dry under a relative humidity of from about 20% to about 80% at a temperature of from about 20° C. to about 37° C. For example, threads can be air-dried for two days at ambient conditions.

Optionally, prior to threads drying, the wet threads can be stretched to a desired length and reduced diameter prior to dying. The stretching can be by either hanging the threads by one end and applying weight to the opposing end, or by horizontally stretching the wet on a surface (either the same or different from the extrusion surface) and adhering the ends to the surface.

### Sterilizing

The threads as described herein can be sterilized using electron beam (e-beam) sterilization methods. Threads as prepared in Example 1 cross-linked with BDDE were washed in a phosphate buffer or Tris buffer solution at pH 10. Some of the solutions further contained 1 mM ascorbic acid, 10 mM ascorbic acid, 100 mM ascorbic acid, 1 M ascorbic acid, 10 mM vitamin E, and 50 mM Na₃PO₄. The threads were then sterilized using standard e-beam techniques at 4 kGy or 20 kGy. In some embodiments, the temperature of the threads can be altered prior to sterilizing. In some embodiments, the temperature is reduced of the threads to about −20° C. In some embodiments, the thread is just below 5° C. after sterilizing.

Using the steps disclosed above, thread can be prepared using any one of the processes disclosed below. As mentioned above, the threads can then be formed into the spheres of the invention.

### Forming the Spherical Forms

As noted above, after forming the threads, the threads (in either a dried, partially dried, or wet state) can then be formed into spherical forms, e.g. spheres, for use in tissue augmentation, in accordance with certain embodiments of the invention.

For example, after threads are formed as described above, the threads may be placed into a suitable device such as a shaker, agitator, or other device that will break up the threads and abrade the fragments to form generally rounded forms which can be the desired spherical forms. For example, extruded threads are fragmented and abraded by operating such a device, e.g. for a sufficient time and at a sufficient frequency, until the threads break up into smaller fragmented threads, and the fragments eventually become rounded spherically shaped forms of a desired size and shape.

In one embodiment, the spherical forms are substantially spherically-shaped. In some embodiments, the spherical forms have a diameter substantially equivalent to a diameter of the thread from which they originated. In other embodiments, the spherical forms have a diameter smaller than the thread from which they originated, which can be accomplished, for example, by subjecting thread fragments to additional agitation and/or abrasion.

It is to be appreciated that the spherical forms of the invention are not limited to forms having a strictly spherical shape. The spherical forms of the invention may include any abraded fragments of threads. The spherical forms typically have a uniform radius, measured from a center of the fragment to all, or at least most, surfaces of the fragment. It can be appreciate that spherical forms may thus be angular when observed with a magnified view.

### Example 1A

### Process 1

Spherical forms can be prepared using the following steps:
1. Cross-linking;
2. Rinsing;
3. Sizing;
4. Drying;
5. Formulating the cross-linked HA with binder;
6. Deaerating;
7. Extruding;
8. Drying;
9. Fragmenting and forming spheres, e.g. by shaking; and
10. Sterilizing.

### Example 1B

### Process 2

Spherical forms can also be prepared using the following steps:
1. Cross-linking;
2. Rinsing;
3. Sizing;
4. Drying;
5. Formulating the cross-linked HA with binder;
6. Deaerating and autoclaving;
7. Extruding;
8. Drying;
9. Fragmenting and forming spheres, e.g. by shaking; and
10. Sterilizing (optional).

### Example 1C

### Process 3

Spherical forms can also be prepared using the following steps:
1. Cross-linking;
2. Extruding;
3. Drying;
4. Rinsing;
5. Drying;
6. Fragmenting and forming spheres, e.g. by shaking; and
7. Auto-claving.

### Example 1D

### Process 4

Spherical forms can also be prepared using the following steps:
1. Cross-linking;
2. Rinsing;
3. Sizing and Formulating the cross-linked HA with binder;
4. Drying;
5. Deaerating;
6. Extruding;
7. Drying;
8. Fragmenting and forming spheres, e.g. by shaking; and
9. Sterilizing.

### Example 1E

### Process 5

Spherical forms can also be prepared using the following steps:
1. Cross-linking;
2. Rinsing;
3. Sizing;
4. Precipitating from ethanol and drying;
5. Formulating the cross-linked HA with binder;
6. Deaerating;
7. Extruding;
8. Drying;
9. Fragmenting and forming spheres, e.g. by shaking; and
10. Sterilizing (optional).

### Example 2

### Washing (Re-Hydrating) and Re-Drying

The dry threads or resulting spherical forms can be washed with an aqueous solvent to remove any contaminants, such as unreacted cross-linking agent. The washing can be performed by various methods, such as submersion in an aqueous solvent or by using a concurrent flow system by placing the threads in a trough at an incline and allowing an aqueous solvent to flow over the threads. In addition, the threads, once it is rehydrated, can be stretched prior to re-drying. The rehydrated and washed threads is then re-dried to provide the dry threads. The re-drying is typically performed under ambient conditions (i.e. ambient temperature and/or pressure) for from about 8 hours to about 24 hours or until the dry thread has a percent hydration of less than about 30%. The threads can be washed several times (e.g. 10 or more times) without losing its structural integrity. Over the course of multiple washing cycles the overall length of the threads can be increased by between about 25% to about 100%.

### Example 3

### Treatment of Wrinkles with Hyaluronic Acid Spherical Forms

Delivery devices can be loaded with spherical forms ranging from diameter of 0.003 in. to 0.05 in. The spherical forms may be e-beam sterilized by NuTek Corp. at 29 kGy. The delivery device then inserts the spherical forms into the skin. The wrinkles to be treated are in the naso-labial fold, peri-orals, peri-orbitals, frontalis (forehead), and glabellar. The delivery device can then insert the spherical forms into the skin at the location of the wrinkle. A plurality of spherical forms can be used to treat the wrinkles in order to achieve the desired fill effect. The wrinkle will be visibly lessened upon spherical form hydration.

### Example 4

### Lip Augmentation

A patient may be implanted with the spherical forms for lip enhancement, either contouring and/or plumping. The patient may receive topical anesthetic on the face, but it is not applied specifically to the lips according to the following procedure:
Peal open the pouch and remove the sterile tray holding the HA (hyaluronic acid) spherical forms or delivery device pre-loaded with hyaluronic acid spherical forms.
Using sterile gloves or a sterile implement such as forceps, remove the desired amount of hyaluronic acid spherical forms from the tray.
Insert the delivery device into one margin of the intended treatment area.
Translate the delivery device within the skin under or near the intended treatment area. If the delivery device is not in a desired location at any point, gently retract the delivery device and reinsert to correct the location.
Implant the desired amount of hyaluronic acid spherical forms into the location.
Withdraw the delivery device and repeat the steps until the desired effect is achieved.

Areas of enhancement include the vermillion border (or white roll) for lip effacement and contouring, the wet-dry mucosal junction for increasing fullness. Other techniques include more diffuse infiltration of the orbicularis oris muscle. The attending clinician is able to select the location of the spherical forms placement, the number of hyaluronic acid spherical forms and the size of the hyaluronic acid spherical forms depending on desired effect. It is contemplated that each area is treated with a plurality of spherical forms wherein each spherical form has a diameter of anywhere from 0.02″ to about 0.025″ when the spherical forms are dry. After hydration, it is contemplated that the spherical forms can have a diameter of from 0.25″ to about 0.5″.

### Example 5

### NMR Study to Determine the Ratio of BDDE Derivative to HA in Cross-Linked Hydrogels

An NMR study was undertaken to determine the ratio of 1,4-butanediol diglycidyl ether derivative (BDDE) to the disaccharide subunit of hyaluronic acid (HA) in cross-linked HA hydrogels. Hydrogels were made at HA concentrations of 8 and 10 percent, cross-linked with 3.2 and 4 percent BDDE, respectively. The gels were rinsed extensively to remove residual BDDE, digested with hyaluronidase, and dried. The resulting powders were dissolved in D₂O and analyzed by proton NMR. The ratio of BDDE to the disaccharide subunit of HA was determined by comparing the peak from the inner methylene hydrogens of 1,4-butanediol at 1.6 ppm to the acetyl methyl group of N-acetylglucosylamine at 2.0 ppm. At equal molar amounts of BDDE and disaccharide subunit these peak areas should integrate to 4 and 3, respectively. The results with the 8% HA hydrogel gave peak areas integrating to 0.75 and 3, respectively, which corresponds to about 0.19 mole of BDDE per mole disaccharide subunit. The results with the 10% HA hydrogel gave peak areas integrating to 0.72 and 3, respectively, which corresponds to about 0.18 mole of BDDE per mole disaccharide subunit.

The experiment with the 8/40 formulation was repeated with second batch of gel and gave results of 0.217 mole of BDDE per mole disaccharide subunit. The average percent BDDE in the 8/40 gel is therefore 20%±2% S.D. Table 5, below, shows additional formulations.

TABLE 5

| | % | | | |
|---|---|---|---|---|
| Gel | BDDE input | % substituted | % cross- | % pendant |
| Formulation | (by wt) | BDDE* | linked BDDE* | BDDE* |
| 12/40 | 40% | 35.2 | 8.5 | 26.7 |
| 12/20 | 20% | 19.1 | 4.9 | 14.2 |
| 12/15 | 15% | 17.2 | 3.5 | 13.7 |

*mol %

What is claimed is:

1. A spherical form for treating skin, the spherical form comprising substantially cross-linked hyaluronic acid.

2. The spherical form of claim 1, wherein the hyaluronic acid is substantially cross-linked with at least about 15 mole % of a butanediol diglycidyl ether (BDDE) derivative relative to the repeating disaccharide unit of the hyaluronic acid, and at least about 5% noncross-linked hyaluronic acid relative to the weight of total hyaluronic acid solids.

3. The spherical form of claim 1, wherein the substantially cross-linked hyaluronic acid is cross-linked with from about 15 mole % to about 25 mole % of the BDDE derivative.

4. The spherical form of claim 1, wherein the substantially cross-linked hyaluronic acid is cross-linked with from about 17 mole % to about 20 mole % of the BDDE derivative.

5. The spherical form of claim 1, wherein the substantially cross-linked hyaluronic acid is cross-linked with at least about 18 weight % of the BDDE derivative relative to the weight of the cross-linked hyaluronic acid.

6. The spherical form of claim 1, wherein the cross-linked hyaluronic acid is present in an amount of from about 60 weight % to about 90 weight % based on the total weight of the spherical form excluding moisture.

7. The spherical form of claim 1, wherein the cross-linked hyaluronic acid is present in an amount of from about 70 weight % to about 80 weight % based on the total weight of the spherical form excluding moisture.

8. The spherical form of claim 1, wherein the noncross-linked hyaluronic acid is present in an amount of from about 10 weight % to about 40 weight % based on the total weight of the spherical form excluding moisture.

9. The spherical form of claim 1, wherein the noncross-linked hyaluronic acid is present in an amount of from about 15 weight % to about 25 weight % based on the total weight of the spherical form excluding moisture.

10. The spherical form of claim 1, wherein the spherical form has a diameter of at least about 0.003 inches.

11. The spherical form of claim 1, wherein the spherical form has a diameter of from about 0.003 inches to about 0.05 inches.

12. The spherical form of claim 1, wherein the spherical form has a diameter of from about 0.004 inches to about 0.03 inches.

13. The spherical form of claim 1, wherein the spherical form has a diameter of from about 0.020 inches to about 0.025 inches.

14. The spherical form of claim 1, wherein the spherical form is formed by fragmenting and abrading a thread form.

15. A method of treating a wrinkle or performing soft tissue augmentation, in a treatment location of a patient in need thereof, said method comprising;
a) inserting a plurality of spherical forms into skin or subcutaneous space of the treatment location, thereby treating the wrinkle or providing the soft tissue augmentation.

16. The method of claim 15, wherein step a) is performed at least 2 or more times in the treatment location.

17. The method of claim 15, further comprising hydrating said plurality of spherical forms.

18. The method of claim 15, wherein the treatment location is selected from the lips, the nasolabial fold, and the tear trough.

19. A kit of parts for use in treating a wrinkle or performing soft tissue augmentation in a patent, said kit comprising said plurality of spherical forms of claim 1.

20. The kit of claim 17, further comprising a means for delivery of said plurality of spherical forms to a patient.
